(19) United States
(12) Patent Application Publication
(10) Pub. No.: US 2021/0338637 A1
(43) Pub. Date: Nov. 4, 2021

(54) INDOLE DERIVATIVES FOR BIOFILM DISRUPTION AND INHIBITION

(71) Applicant: LIFE MATTERS LTD., Moshav Hazav (IL)

(72) Inventors: Ariel KUSHMARO, Beer Yaakov (IL); Robert S. MARKS, Omer (IL); Karina GOLBERG, Beer Sheva (IL)

(21) Appl. No.: 17/375,703

(22) Filed: Jul. 14, 2021

Related U.S. Application Data
(63) Continuation-in-part of application No. 15/862,040, filed on Jan. 4, 2018, now Pat. No. 11,096,925.
Application No. 15/862,040 is a continuation-in-part of application No. PCT/IL2016/050732, filed on Jul. 7, 2016.

(30) Foreign Application Priority Data
Jul. 7, 2015 (SG) 10201505353S

(51) Int. Cl. A61K 31/404; A61K 9/00; A61P 31/04

(52) U.S. Cl. CPC A61K 31/404; A61P 31/04; A61K 9/0024

(57) ABSTRACT

A method for inhibiting biofilm formation by bacteria on a surface or disrupting existing biofilm on a surface, comprising contacting said surface or existing biofilm with at least one compound selected from the group consisting of 2-(indolin-2-yl)-1H-ind, di(1H-indol-3-yl)methane and 1,1′-biindole, is provided.

### CROSS-REFERENCE TO RELATED APPLICATIONS

The present application is a Continuation-in-Part of U.S. application Ser. No. 15/862,040 filed Jan. 4, 2018, which is a Continuation-in-Part of Application No. PCT/IL2016/050732 filed Jul. 7, 2016, in which the United States is designated, and claims the benefit of priority from Singapore Application No. 102015053535 filed Jul. 7, 2015, the entire contents of each and all applications being hereby incorporated by reference herein in their entirety as if fully disclosed herein.

### FIELD OF THE INVENTION

The present invention relates in general to prevention of biofilm formation and disruption of existing biofilm.

### BACKGROUND OF THE INVENTION

In general, bacteria may exist as single, independent cells (planktonic) or they may be organized into sessile aggregates. The latter form is commonly referred to as the biofilm growth phenotype. Acute infections often involve planktonic bacteria, which are generally treatable with antibiotics, whereas infections involving biofilm-residing bacteria often turn out to be untreatable and will develop into a chronic state. It has been estimated that most bacterial infections in humans are correlated with biofilm and about 50% of the nosocomial infections are indwelling devices-associated. The high cell densities typical in mature biofilms provide the biofilm formers with a first line of defense by preventing penetration of the biofilm by competitors. Accordingly, reducing the cell densities in the biofilm will aid in the treatment of the bacterial infection.

The ability of many bacteria to adhere to surfaces and to form biofilms has also major implications in a variety of industries including shipping, energy, water, food (e.g. dairy, fish, poultry, meat, and Ready-To-Eat food processing), oil drilling, paper production, marine aquaculture, etc.

Existing methods rely primarily on coating devises and submerged surfaces with a protecting coat, and there is no satisfactory method available for treating medically important biofilm. There is thus a pressing need for novel methods for preventing biofilm formation and disrupting existing biofilm in medical and environmental settings.

### SUMMARY OF INVENTION

In one aspect, the present invention provides a compound selected from 2-(indolin-2-yl)-1H-indole (compound of formula I), di(1H-indol-3-yl)methane (compound of formula II) and 1,1′-biindole (compound of formula III), or a combination thereof, for use in inhibiting biofilm formation by bacteria on a surface or disrupting existing biofilm on a surface.

In another aspect, the present invention is directed to a compound selected from a compound of formula (I), (II) and (III), or any combination thereof, for use in reducing bacterial virulence.

In a further aspect, the present invention provides a composition comprising a compound selected from a compound of formula (I), (II) and (III), or any combination thereof.

In an additional aspect, the present invention provides a method for inhibiting biofilm formation by bacteria on a surface or disrupting existing biofilm on a surface, comprising contacting said surface or existing biofilm with an effective amount of at least one compound selected from a compound of formula (I), (II) and (III).

In some embodiments, the at least one compound is a compound of formula (I) and/or a compound of formula (III).

In yet another aspect, the present invention is directed to a medical device coated, washed or rinsed with a compound selected from a compound of formula (I), (II) and (III), or any combination thereof.

In an additional aspect, the present invention is directed to a pharmaceutical composition comprising 2-(indolin-2-yl)-1H-indole and/or 1,1′-biindole, and a pharmaceutically acceptable carrier.

In some embodiments, the pharmaceutical composition is a gel, a stick pill, a rinsing liquid, a toothpaste, a tablet, a topical medicament, an oral dentifrice, an injectable composition, an oral tablet, a lozenge, a soft gelatin capsule or an aerosol spray.

In some embodiments, the pharmaceutical composition further comprises at least one antibiotic or antibacterial agent.

In yet an additional aspect, the present invention is directed to a method for inhibiting formation of biofilm by bacteria on a surface or disrupting existing biofilm formed by bacteria on a surface, comprising contacting said surface or said existing biofilm with an effective amount of a pharmaceutical composition comprising 2-(indolin-2-yl)-1H-indole and/or 1,1′-biindole, wherein the surface is a surface of a mammalian cell, tissue, organ, system, or structure.

In some embodiments, the bacteria are gram-negative bacteria. In some embodiments, the bacteria are gram-positive bacteria.

In some embodiments, the surface is a surface of a cell, tissue, or structure of the gastrointestinal tract, the respiratory system, the skin, or of an epithelium. In some embodiments, the surface is a surface of a medical device intended for insertion into a subject's body, such as a pacemaker, pacemaker leads, catheter or stent.

In some embodiments, the method is for prophylaxis, metaphylaxis, or therapy or an infectious disease caused by said bacteria in said biofilm.

In some embodiments, the method further comprises contacting said surface with at least one antibiotic or antibacterial agent.

In still an additional aspect, the present invention is directed to a medical device coated, washed, or rinsed with a pharmaceutical composition comprising 2-(indolin-2-yl)-1H-indole and/or 1,1′-biindole.

In some embodiments, the medical device is a pacemaker, pacemaker leads, catheter or stent.

In still a further aspect, the present invention is directed to a method for inhibiting formation of biofilm by gram-positive bacteria on a surface or disrupting existing biofilm formed by gram-positive bacteria on a surface, comprising contacting said surface or said existing biofilm with an effective amount of at least one compound selected from the group consisting of 2-(indolin-2-yl)-1H-ind, di(1H-indol-3-yl)methane, and 1,1′-biindole, wherein the surface does not include a surface in an oral cavity of a mammal.

In some embodiments, the surface is a surface of a cell, tissue, or structure of the gastrointestinal tract, the respiratory system, the skin, or of an epithelium.

In some embodiments, the method is for prophylaxis, metaphylaxis or therapy of an infectious disease caused by said bacteria in said biofilm, wherein said bacteria do not include Paenibacillus larvae.

In some embodiments, the method further comprises contacting the surface or existing biofilm with an antibiotic or an antibacterial agent.

In some embodiments, the surface is a surface of a plant cell, tissue or structure. In some embodiments, the surface is a surface of a medical device intended for insertion into a subject's body, such as a pacemaker, pacemaker leads, catheter or stent. In some embodiments, the surface is a surface intended for contact with water or an aqueous solution, such as a surface of a ship hull, a pipe, a filter, a strain or a pump.

In some embodiments, the method inhibits biofilm formation by gram-positive bacteria on a surface of a submerged object, or disrupting existing biofilm formed by gram-positive bacteria on a surface of a submerged object, such as a surface of a ship hull, a pipe, a filter, a strain or a pump.

In some embodiments, the Gram-positive bacteria are selected from Streptococcus, Staphylococcus, Corynebacterium, Listeria, Bacillus, Clostridium, Catabacter hongkongensis, Mycobacterium, Mycoplasma, Enterococci, and Actinomyces.

### BRIEF DESCRIPTION OF DRAWINGS

FIGS. 1A-1B show prevention of biofilm formation. Confocal scanning laser micrographs (CSLM) of P. aeruginosa PA01 (A) and A. baumannii (B) formed in glass-bottomed 96 well plates after 18 h of static incubation at 37° C. Cultures were grown in the presence of either 50 μM DIV or NN, or an equivalent amount of DMSO for control. Biofilms were stained using the Live/Dead bacterial viability kit. Live, dead and total bio-volumes (μm³/μm²) were calculated based on image analysis and data from the IMARIS software. Bars indicate standard deviations for triplicate sets of experiments.

FIG. 2 depicts destruction of P. aeruginosa PA01 mature biofilm; biofilm formed in a flow cell after 72 h of incubation at 37° C. and 48 h more in the presence of 50 μM NN, 20 μg/ml Tobramycin, and 50 μM NN with 20 μg/ml tobramycin. Biofilms were stained with Live/Dead bacterial viability kit. Quantification of BioVolume; Live, dead and total bio-volumes (μm³/μm²) calculated based on image analysis and data from the IMARIS software. The images were acquired from three different areas in each treatment.

FIGS. 3A-3B show effect on, P. aeruginosa PA01 pathogenesis. (A) Inhibition of virulence factors production in P. aeruginosa PA01, which were grown in the presence of 50 μM NN or DIV. Tetracycline is used as positive control. Results are based on OD measurements distinctive to each factor and normalized to bacterial growth at OD600 nm. Error bars represent SD of three independent repetitions. (B) C. elegans killing assay assed by SYTOX Orange stain within 24 h.

FIGS. 4A-4B show reduced adhesion and virulence of P. aeruginosa PA01 in human A549 lung cells model. (A) Cytotoxicity effect and apoptosis of A549 cells by P. aeruginosa PA01 pre-treated with DIV or NN during 24 h. The infection progress was monitored by calcein staining using Operetta screening system. All the experiments were performed in triplicates. (B) Adhesion of P. aeruginosa PA01 (5×10⁷ CFU/ml) pre-treated with DIV or NN to A549 cells for 1 hr of incubation. Excess bacteria were removed and the released cells were plated followed CFU counts determination.

FIGS. 5A-5D show biofilm attenuation of the pathogens P. stuartii (A), S. marcescens (B), A. baumannii (C) and P. aeruginosa PA01 (D). Cultures were grown in the presence of either 50 μM DIM or an equivalent amount of DMSO for control. Biofilms were stained with the LIVE/DEAD bacterial viability kit. For each species and per treatment, live, dead and total bio-volumes (μm³/μm²) were calculated based on image analysis and data from the IMARIS software, and % biofilm inhibition was calculated based on live bio-volumes. The results are the average values of analysis of at least three micrographs.

FIG. 6 depicts destruction of mature, differentiated biofilm of P. aeruginosa PA01. Statistical analysis of CSLM images of biofilm formed in a flow system after 120 h, and with supplementation of 50 μM DIM, 20 μg/ml Tobramycin and a combined treatment of 50 μM DIM with 20 μg/ml Tobramycin. Biofilms were stained with the LIVE/DEAD bacterial viability kit. Quantification of bio-volume: live, dead and total bio-volumes (μm³/μm²) were calculated based on image analysis and data from the IMARIS software.

FIG. 7 shows inhibition of virulence factor production in P. aeruginosa PA01 that was grown in the presence of 50 μM DIM or 0.6 μg/ml tetracycline treatment as a positive control. Results are based on OD measurements specific to each factor and normalized to the growth OD of 600 nm. Bars indicate standard deviations for triplicate sets of experiments.

FIG. 8 shows prevention of biofilm formation by Erwinia carotovora. Biofilm formed in glass-bottomed 96 well plates after 18 h of static incubation at 37° C. was investigated by CSLM. Cultures were grown in the presence of either 50 μM DIM or NN, or an equivalent amount of DMSO for control. Biofilms were stained using the Live/Dead bacterial viability kit. Live, dead and total bio-volumes (μm³/μm²) were calculated based on image analysis and data from the IMARIS software.

FIGS. 9A-9C show inhibition of gram positive bacterial biofilm formation. A. CSLM of Clostridium perfringens and Staphylococcus aureus (MRSA) formed during 48 h of continuous flow in a flow cell system. Cultures were grown in the presence of either 50 μM DIM or an equivalent amount of DMSO (control). B, C. Bar graphs showing the bio-volume of live (black) and dead (gray) bacteria and % inhibition (empty bars) in the control (left group of bars) and DIM-treated (right group of bars) bacteria for Clostridium perfringens (B) and Staphylococcus aureus (MRSA, C). Biofilms were stained using the LIVE/DEAD™ BacLight™ bacterial viability staining kit (Molecular Probes Inc., Eugene, Oreg., USA). Live and dead bio-volumes (μm³/μm²) were calculated based on image analysis and data from the IMARIS cell imaging software, and % biofilm inhibition was calculated based on live bio-volumes. Bars indicate standard deviations for triplicate sets of experiments. Asterisks indicate significant differences compared to control (independent samples t-test; *p<0.05; **p<0.01; ***p<0.001).

### DETAILED DESCRIPTION OF THE INVENTION

A structured consortium attached on a living or inert surface formed by microbial cells and surrounded by the self-produced extracellular polymeric matrix is known as biofilm. Biofilms are thus defined as microbially derived sessile communities characterized by cells that are irreversibly attached to a substratum or interface or to each other, are embedded in a matrix of extracellular polymeric substances that they have produced, and exhibit an altered phenotype with respect to growth rate and gene transcription. A typical development of biofilm—taking Pseudomonas aeruginosa as an example—includes several stages, i.e., attachment to a surface; formation of microcolonies; development of young biofilm; differentiation of structured mature biofilm, and dispersal of mature biofilm. Pathogenic bacteria residing in biofilms can cause chronic infections, and aggressive and intensive antibiotic treatment is usually helpful to control the exacerbations of such infections induced by dispersed bacteria and reduce the biofilms, but cannot eradicate the biofilm infections, because the minimal concentration of antibiotic for eradication of mature biofilm is difficult to reach in vivo. Therefore, once a bacterial biofilm infection is established, it becomes difficult to eradicate. Bacterial biofilm formation is widely found in natural environments with water, and also in human diseases, especially in patients with indwelling devices for the purpose of medical treatments (Wu et al., 2014).

The inventors of the present invention have screened over 100 bacterial isolates obtained from several coral species for their anti-biofilm activity and abilities to inhibit quorum sensing using different bioreporter strains. The present invention is based on the finding that two compounds identified in the screen as 1,1′-Biindole (hereinafter, “NN”) (CAS Registry Number 479500-92-0) 2-(indolin-2-yl)-1H-indole (hereinafter, “DIV”) (CAS Registry Number 38505-89-4) were found to inhibit biofilm formation, attenuate bacterial virulence and disassemble or reduce existing biofilm. NN was first prepared and characterized by Zhang et al. (2011). DIV was first prepared and characterized by Somei et al., (1997). In addition, it was found that the anti-cancer compound di(1H-indol-3-yl)methane (hereinafter, “DIM”) (CAS Registry Number 1968-05-4; WO 98/50357) has similar properties. DIM has further been shown to have antibiotic activity against P. larvae as determined by agar diffusion method (Brenda et al., 2010), to have antifouling activity (US 2016/0037773), to be an immune response activator (US 2006/0100264) and to be useful for the prevention and or treatment of neurological conditions (WO 2005/016339).

In view of the above, in one aspect, the present invention provides at least one compound selected from 2-(indolin-2-yl)-1H-indole, di(1H-indol-3-yl)methane, and 1,1′-biindole, for use in inhibiting biofilm formation by bacteria on a surface or disrupting existing biofilm formed by bacteria on a surface.

In some embodiments, the at least one compound is 2-(indolin-2-yl)-1H-indole and/or 1,1′-biindole.

In some embodiments, the at least one compound is 2-(indolin-2-yl)-1H-indole and 1,1′-biindole.

TABLE 1
Structures I, II and III

| Compound of | Compound of | Compound of |
|---|---|---|
| formula I | formula II | formula III |
| 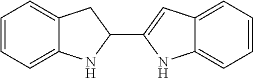 [SMILES: c1ccc2c(c1)CC(c1cc3ccccc3[nH]1)N2] | 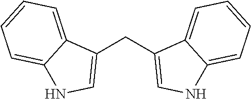 [SMILES: c1ccc2c(Cc3c[nH]c4ccccc34)c[nH]c2c1] | 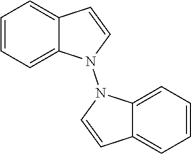 [SMILES: c1ccc2c(c1)ccn2-n1ccc2ccccc21] |
| 2-(indolin-2-y1)-1H-indole | di(1H-indo1-3-yl)methane | 1,1′-Biindole |
| (DIV) | (DIM) | (NN) |

The terms “disrupting”, “disassembling”, “reducing” and “eradicating” are used interchangeably herein to describe disappearance of an existing biofilm at a rate that is greater than an untreated biofilm or a biofilm treated with a compound known to have no effect on biofilm stability.

In another aspect, the present invention is directed to a method for inhibiting biofilm formation by bacteria on a surface or disrupting existing biofilm formed by bacteria on a surface, comprising contacting said surface or said existing biofilm with an effective amount of at least one compound selected from a compound of formula (I), a compound of formula (II), and a compound of formula (III).

In some embodiments, the at least one compound is 2-(indolin-2-yl)-1H-indole (compound of formula (I)) and/or 1,1′-biindole (compound of formula (III)).

In some embodiments, the at least one compound is 2-(indolin-2-yl)-1H-indole (compound of formula (I)) and 1,1′-biindole (compound of formula (III)).

The surface may be a biotic (living) surface or an abiotic (not living) surface.

Biotic surfaces include surfaces of living organisms including, e.g. mammals (such as humans), plants, poultry, fish, etc. The surfaces may be surfaces of cells, tissues, organs, systems, structures, cavities, etc. of living organisms, such as mammals (e.g. humans).

In some embodiments the surface is a surface of a mammalian body. In some embodiments, the surface is a surface of a human body.

Specific relevant surfaces of living mammals (e.g. humans) that may be treated by the present invention include surfaces that are in contact with air or fluids such as surfaces of the respiratory system, the gastrointestinal tract, the skin, eyes, ears, the urogenital system, and epithelium of the above systems.

More specifically, such surface may be a surface of an open wound, ulcer, or lesion in any part of the body, such as skin or epithelium, or in a certain part of the gastrointestinal tracts such as large intestines, small intestine, duodenum, jejunum, ileum, rectum, esophagus, colon, certain part of the respiratory system such as nose, lungs, bronchioles, sinuses, pharynx, trachea. Additional specific surfaces relevant for the invention are of the cornea, the outer ear, the ear canal, vascular and lymph endothelium, endocardium, bile duct, urinary tract, urinary bladder.

In certain embodiments, the surface is a surface of a mammalian, poultry or fish cell, tissue or structure. For example, the cell or tissue may be lung, muscle or skin cell or tissue, and the structure is a tooth.

In some embodiments, the surface is a surface of a mammalian cell, tissue, organ, system, or structure.

In some embodiments, the surface is a surface of a cell, tissue, or structure of the gastrointestinal tract, the respiratory system, the skin, or an epithelium. In some embodiments, the surface is a surface of a cell, tissue, or structure of the gastrointestinal tract, the respiratory system, the skin, or an epithelium of a human body.

In some embodiments, the surface does not include a surface in a mouth. In some embodiments, the surface does not include a surface in an oral cavity.

In some embodiments, the surface does not include a surface in a mouth, or a surface in an oral cavity of a mammal.

A surface in a mouth or oral cavity includes, for example, a surface of a tooth, a tongue, a gum, oral mucosa, or periodontal pocket.

In some embodiments, the surface does not include a surface of a gum. In some embodiments, the surface does not include a surface of a tooth. In some embodiments, the surface does not include a surface of a periodontal pocket.

In some embodiments, the tissue or structure does not include the mouth. In some embodiments, the tissue or structure does not include the oral cavity. In some embodiments, the tissue or structure does not include oral mucosa. In some embodiments, the tissue or structure does not include a gum. In some embodiments, the tissue or structure does not include a tooth. In some embodiments, the tissue or structure does not include a periodontal pocket.

In certain embodiments, the compounds are for use in prophylaxis, metaphylaxis or therapy of an infectious disease caused by the bacteria in the biofilm.

In certain embodiments, the compounds are for use in prophylaxis, metaphylaxis or therapy of an infectious disease caused by the bacteria when present in biofilm adherent to the cell, tissue or structure surface resulting from said inhibiting biofilm formation or disrupting existing biofilm.

In certain embodiments, the methods are for prophylaxis, metaphylaxis or therapy of an infectious disease caused by the bacteria in the biofilm.

In certain embodiments, the methods are for prophylaxis, metaphylaxis or therapy of an infectious disease caused by the bacteria when present in biofilm adherent to the cell, tissue or structure surface resulting from said inhibiting biofilm formation or disrupting existing biofilm.

In certain embodiments, the bacteria do not include Paenibacillus larvae.

Examples for such infectious diseases suitable for treating according to the present invention include, but are not limited to contact lens-associated microbial keratitis (CLMK), chronic otitis, implant infection, chronic skin and lung infection, burn-related infection, intravascular catheter infection, prosthetic valve endocarditis, pacemaker infection, endocarditis, biliary stent infection, peritoneal dialysis catheter infection, prosthetic joint infection, urinary stent infection, intravascular stent infection, different implants-associated infection, pulmonary infection, stomach and gastrointestinal infection.

In some embodiments, the compounds or the methods of the invention are not for treating oral mucosal disorders such as periodontitis and gingivitis.

In some embodiments, the infectious disease is not a periodontal disease. In some embodiments, the infectious disease is not periodontitis or gingivitis.

Abiotic surfaces include surfaces such as those of devices, e.g. medical device, as well as other surfaces that are in touch with water or humidity. Examples for surfaces often exposed to water include submerged surfaces such as ship hulls, boat propellers, cages, underwater dock structures, underwater structures on offshore oil platforms, submarine mines, buoys, submarine cables, cooling systems of power plants, pipes and filters of plants, such as desalination plants, industrial or portable water system piping, natural aquatic systems.

In certain embodiments, the surface is a surface of a medical device intended for insertion into a subject's body, i.e. the compounds and the methods of the present invention may be used in inhibiting biofilm formation by bacteria on a surface of a medical device intended for insertion into a subject's body or disrupting existing biofilm on a surface of a medical device intended for insertion into a subject's body.

In still an additional aspect, the present invention is directed to a medical device intended for insertion into a subject's body, wherein said medical device is coated with at least one compound selected from a compound of formula (I), a compound of formula (II) and a compound of formula (III).

In still a further aspect, the present invention is directed to a medical device intended for insertion into a subject's body, wherein said medical device is coated with at least one compound selected from a compound of formula (I) and/or a compound of formula (III).

The term “medical devise intended for insertion into a subject's body” as used herein refers to surgically invasive devices or implantable devices as defined, e.g. but not limited to the European Commission DG Health and Consumer Directorate B, Unit B2 “Cosmetics and medical devices” Guidelines Relating to the Application of the Council Directive 93/42/EEC on Medical Devices.

In certain embodiments, the medical devise intended for insertion into a subject's body is a surgically invasive devices intended for short-term use (>60 minutes, <30 days), such as, but not limited to, clamps, infusion cannulae, skin closure devices, temporary filling materials, tissue stabilizers used in cardiac surgery, cardiovascular catheters, cardiac output probes, temporary pacemaker leads, thoracic catheters intended to drain the heart, including the pericardium, carotid artery shunts, ablation catheter, neurological catheters, cortical electrodes or brachytherapy devices.

In certain embodiments, the medical device intended for insertion into a subject's body is an implantable device or long-term surgically invasive device (>30 days), such as prosthetic joint replacements, ligaments, shunts, stents and valves (e.g. pulmonary), nails and plates, intra-ocular lenses, internal closure devices (including vascular closure devices), tissue augmentation implants, peripheral vascular catheters, peripheral vascular grafts and stents, penile implants, non-absorbable sutures, bone cements and maxillo-facial implants, visco-elastic surgical devices intended specifically for ophthalmic anterior segment surgery, bridges and crowns, dental filling materials and pins, dental alloys, ceramics and polymers, prosthetic heart valves, aneurysm clips, vascular prosthesis and stents, central vascular catheters, spinal stents, CNS electrodes, cardiovascular sutures, permanent and retrievable vena cava filters, septal occlusion devices, intra-aortic balloon pumps, external left ventricular assisting devices.

Additional medical devices relevant to the invention include contact lenses and hearing aids such as cochlear implants.

In particular, the surface is a surface of a pacemaker, pacemaker leads, catheter or stent.

The ability of many bacteria to adhere to surfaces and to form biofilms has also major implications in a variety of industries including shipping, energy, water, food (e.g. dairy, fish, poultry, meat, and Ready-To-Eat food processing), oil drilling, paper production, marine aquaculture, etc.

In the case of the food processing industry, biofilm causes chronic bacterial contamination in food processing equipment such as pasteurization pipes and tubes.

In the case of marine-based industries, marine fouling is typically described as comprising several stages, with the early step of bacterial adhesion initiating the formation of a biofilm, which is then followed by secondary colonizers of spores of macroalgae (e.g. Enteromorpha intestinalis, ulothrix) and protozoans (e.g. vorticella, zoothamnium sp.) that attach themselves. Lastly, tertiary colonizers—the macrofoulers attach including tunicates, mollusks and sessile Cnidarians. Thus, biofilm formation provides a substratum for biofouling of submerged surfaces such as ship hulls, boat propellers, cages, underwater dock structures, underwater structures on offshore oil platforms, submarine mines, buoys, submarine cables, cooling systems of power plants, pipes and filters of plants, such as desalination plants, industrial or portable water system piping, natural aquatic systems etc.

In view of the above, in certain embodiments, the compounds described above may be for use in inhibiting biofilm formation by bacteria on a surface intended for contact with water or an aqueous solution (e.g. milk or any other liquid food processed in the food industry), such as a the surface of ship hulls, boat propellers, cages, underwater dock structures, underwater structures on offshore oil platforms, submarine mines, buoys, submarine cables, cooling systems of power plants, pipes, filters, strains or pumps; i.e. the method of the present invention may be employed in inhibiting biofilm formation by bacteria on a surface of a submerged object or disrupting existing biofilm on such a surface of a submerged object.

The aerial tissues of plants are colonized by a wide variety of bacteria, fungi, and yeasts. These colonizing microorganisms are known as epiphytes. Bacteria are the primary colonizers of leaf surfaces, some of which are spoilage bacteria and some of which are pathogenic bacteria, such as Campylobacter jejuni, E. coli 0157:H7, Salmonella spp., Shigella spp., Listeria monocyrogenes, Clostridium botulinum, Campylobacter, and Bacillus cereus. These bacteria are also present on other aerial tissues such as flowers and fruits. Furthermore, pathogenic bacteria may adhere to leaves, fruit and vegetables and form biofilm on their surfaces.

It has been found in accordance with the present invention that application of the compound NN and DIM prevents biofilm formation by the bacteria Erwinia carotovora known to infect a variety of vegetables and plants including carrots, potatoes, cucumbers, onions, tomatoes, lettuce and ornamental plants like iris (Example 3).

Thus, in certain embodiments, the compounds described above may be for use in inhibiting biofilm formation by bacteria on a surface of a plant or plant part, or a cell, tissue or structure or the plant or plant part. The plant part may be a fruit, a root, a seed, a stalk, a flower, a leaf, or any other plant part. The plant part may be a post-harvest fresh product such as a fruit or vegetable.

In certain embodiments the plant may be, but is not limited to, a plant producing carrots, potatoes, cucumbers, onions, tomatoes, lettuce, apples, citrus fruit or plums.

In certain embodiments, the plant cell is derived from and the tissue or structure is selected from a leaf, a root, a flower, a fruit, or other edible structures of a plant.

The biofilm dwelling bacteria subject of the present invention may be any bacteria, i.e. Gram-negative or Gram-positive bacteria or mycoplasma and spiroplasma. Within these groups there are bacteria that associate with animal cells, plant cells or artificial surfaces.

In certain embodiments, the bacteria of the present invention, i.e. bacteria producing, forming and/or residing in the biofilms discussed herein are Gram-negative bacteria.

The term “Gram-negative bacteria” as used herein refers to bacteria displaying the following characteristics: An inner cell membrane is present (cytoplasmic); A thin peptidoglycan layer is present (This is much thicker in gram-positive bacteria); Has outer membrane containing lipopolysaccharides (LPS, which consists of lipid A, core polysaccharide, and O antigen) in its outer leaflet and phospholipids in the inner leaflet; Porins exist in the outer membrane, which act like pores for particular molecules; Between the outer membrane and the cytoplasmic membrane there is a space filled with a concentrated gel-like substance called periplasm; The S-layer is directly attached to the outer membrane rather than to the peptidoglycan; If present, flagella have four supporting rings instead of two; Teichoic acids or lipoteichoic acids are absent; Lipoproteins are attached to the polysaccharide backbone; Some contain Braun's lipoprotein, which serves as a link between the outer membrane and the peptidoglycan chain by a covalent bond; Most, with very few exceptions, do not form spores.

Examples of Gram-negative bacteria, the biofilms of which can be treated in accordance with the present invention, are, but are not limited to, Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella, etc. Other notable groups of gram-negative bacteria include the cyanobacteria, spirochaetes, green sulfur, and green non-sulfur bacteria.

In some embodiments, the bacteria are not Porphyromonas gingivalis.

Medically relevant gram-negative cocci include the four organisms that cause a sexually transmitted disease (Neisseria gonorrhoeae), meningitis (Neisseria meningitidis), and respiratory symptoms (Moraxella catarrhalis, Haemophilus influenzae).

Medically relevant gram-negative bacilli include a multitude of species. Some of them cause primarily respiratory problems (Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi).

Gram-negative bacteria associated with hospital-acquired infections include Acinetobacter baumannii, which cause bacteremia, secondary meningitis, and ventilator-associated pneumonia in hospital intensive-care units.

Bacterial adhesion of e.g. the Gram-negative bacteria Vibrio alginolyticus, Pseudomonas putrefaciens or cyanobacteria initiates the formation of biofilm formation as a primary step in marine fouling.

Plant pathogenic Gram-negative bacteria are classified within the Phylum Proteobacteria. The principal genera of plant Gram-negative pathogenic bacteria are Agrobacterium, Erwinia, Pseudomonas, Xanthomonas and Xylella. [Plant diseases—Britannica Online Encyclopedia]

In certain embodiments, the Gram-negative bacterial species is selected from Pseudomonas aeruginosa, Acinetobacter baumannii, Serratia marcescens, Providencia stuartii, Erwinia carotovora, and Helicobacter pylori.

In certain embodiments, the bacteria of the present invention, i.e. bacteria producing, forming, and/or residing in the biofilms discussed herein above are Gram-positive bacteria.

The term “Gram-positive bacteria” as used herein refers to bacteria displaying the following characteristics: a cytoplasmic lipid membrane; a thick peptidoglycan layer; lipoteichoic acids in the cell wall formed from teichoic acids and lipoids and serving as chelating agents and also for certain types of adherence; peptidoglycan chains cross-linked to form rigid cell walls by a bacterial enzyme DD-transpeptidase; and a much smaller periplasmic volume than that of gram-negative bacteria. Gram-positive bacteria also have a surface layer called “S-layer” as in gram-negative bacteria, which is here attached to the peptidoglycan layer. Some species have a capsule, usually consisting of polysaccharides, and some species have flagella.

Six gram-positive genera are typically pathogenic in humans. Two of these, Streptococcus and Staphylococcus, are cocci (sphere-shaped). The remaining organisms are bacilli (rod-shaped) and can be subdivided based on their ability to form spores. The non-spore formers are Corynebacterium and Listeria (a coccobacillus), whereas Bacillus and Clostridium produce spores. The spore-forming bacteria can again be divided based on their respiration: Bacillus is a facultative anaerobe, while Clostridium is an obligate anaerobe. Gram-positive bacteria are capable of causing serious and sometimes fatal infections in newborn infants. Novel species of clinically relevant gram-positive bacteria also include Catabacter hongkongensis, which is an emerging pathogen belonging to Firmicutes.

Examples of Gram-positive bacteria, the biofilms of which can be treated in accordance with the present invention, are, but are not limited to, Streptococcus, Staphylococcus, Corynebacterium, Listeria, Bacillus, Clostridium, Catabacter hongkongensis, Mycobacterium, Mycoplasma, Enterococci, and Actinomyces.

In certain embodiments, the Gram-positive bacterial species is selected from Clostridium perfringens and Staphylococcus aureus.

It is noted that although certain embodiments relate to gram-negative bacteria or to gram-positive bacteria, the biofilm in which they reside, or which is formed by such bacteria, may also include additional types of microorganisms, including additional types of bacteria.

Accordingly, in some embodiments, when the invention relates to inhibiting formation of biofilm by gram-negative bacteria, or disrupting existing biofilm formed by gram-negative bacteria, the biofilm also includes gram-positive bacteria.

Additionally, in some embodiments, when the invention relates to inhibiting formation of biofilm by gram-positive bacteria, or disrupting existing biofilm formed by gram-positive bacteria, the biofilm also includes gram-negative bacteria.

In some embodiments, the biofilm is a pathogenic biofilm. In some embodiments, the biofilm is a non-pathogenic biofilm.

In some embodiments, the biofilm of the invention includes fewer than 100 different bacterial species. In some embodiments, the biofilm includes fewer than 90, 80, 70, or 60 different bacterial species. In some embodiments, the biofilm includes fewer than 50 different bacterial species. In some embodiments, the biofilm includes fewer than 40, 30, or 20 different bacterial species. In some embodiments, the biofilm includes 10 different bacterial species or fewer. In some embodiments, the biofilm includes 5 different bacterial species or fewer. In some embodiments, the biofilm includes 3 different bacterial species or fewer. In some embodiments, the biofilm includes a single bacterial species.

In certain embodiments, the compounds used in accordance with the present invention are for use in combination with at least one antibiotic or antibacterial agent.

In certain embodiments, the methods of the present invention further include administration of at least one antibiotic or antibacterial agent.

In some embodiments, the antibiotic or antibacterial agent is administered together with the at least one compound of the invention. In some embodiments, the antibiotic or antibacterial agent is administered before administration of the at least one compound of the invention. In some embodiments, the antibiotic or antibacterial agent is administered after administration of the at least one compound of the invention.

In certain embodiments, the pharmaceutical compositions of the present invention further include at least one antibiotic or antibacterial agent.

The term “antibiotic” is used interchangeably herein with the term “antibacterial” and refers to a compound that kills or inhibits the growth of bacteria but have no effect on biofilm formation or eradication. Any commercially available antibiotic compound can be used and is chosen by the skilled artisan according to its efficacy against the intended target bacteria.

Classes of antibiotics include aminoglycosides, ansamycins, beta-lactams, carbapenems, cephalosporins, DHFR inhibitor, fluoroquinolones, glycopeptides, ketolides, lincosamides, lipoglycopeptides, lipopeptides, macrolides, monobactams, nitroimidazoles, nitrofurans, oxazolidinones, penicillins, pleuromutilins, polypeptides, rifamycins, streptogramins, sulfonamides, and tetracyclines.

Additional antibiotics that do not fit into the classes listed above include, e.g., chloramphenicol, clindamycin, daptomycin, fosfomycin, lefamulin, metronidazole, mupirocin, and tigecycline.

In certain embodiments, the antibiotic compound is an aminoglycoside, such as Tobramycin, Kanamycin A, Amikacin, Dibekacin, Gentamicin, Sismicin, Netilmicin, Neomycin B, Neomycin C, Neomycin E, Streptomycin, and Spectinomycin(Bs); an ansamycin, such as Geldanamycin, Herbimycin, and Rifaximin; a carbapenem, such as Imipenem, Meropenem, Ertapenem, Doripenem, Panipenem/betamipron, Biapenem, and Tebipenem; a cephalosporin, such as cefaclor, cefprozil, and cefuroxime; a glycopeptide, such as Vancomycin, Teicoplanin, Telavancin, Ramoplanin, Dalbavancin, Oritavancin, and Decaplanin; a lincosamide, such as Clindamycin and Lincomycin; a lipopeptide, such as Daptomycin; a macrolide, such as Azithromycin, Clarithromycin, Erythromycin, Fidaxomicin, Telithromycin, Carbomycin A, Josamycin, Kitasamycin, Midecamycin/midecamycin acetate, Oleandomycin, Solithromycin, Spiramycin, Troleandomycin, Tylosin/tylocine, and Roxithromycin; a ketolide, such as Telithromycin, Cethromycin, and Solthromycin; a monobactam, such as Aztreonam, Tigemonam, Nocardicin A, and Tabtoxin; a nitrofuran, such as Difurazone (also known as Nitrovin), Furazolidone, Nifurfoline, Nifuroxazide, Nifurquinazol, Nifurtoinol, Nifurzide, Nitrofural (also known as nitrofurazone), Nitrofurantoin, Ranbezolid, Furaltadone—an antiprotozoal, Furazidine, Furylfuramide, Nifuratel, and Nifurtimox; an oxazolidinone, such as Linezolid, Posizolid, Tedizolid, Radezolid, Cycloserine, and (S)-5-((isoxazol-3-ylamino)methyl)-3-(2,3,5-trifluoro-4-(4-oxo-3,4-dihydropyridin-1(2H)-yl)phenyl)oxazolidin-2-one; a penicillin, such as Penicillin G, Penicillin K, Penicillin N, Penicillin O, Penicillin V, Methicillin, Nafcillin, Oxacillin, Cloxacillin, Dicloxacillin, Flucloxacillin, Ampicillin, Amoxicillin, Pivampicillin, Hetacillin, Bacampicillin, Metampicillin, Talampicillin, Epicillin, Carbenicillin, Ticarcillin, Temocillin, Mezlocillin, Piperacillin, Clavulanic acid, Sulbactam, and Tazobactam; a polypeptide, such as actinomycin, bacitracin, colistin, and polymyxin B; a fluoroquinolone, such as Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin; a sulfonamide, such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfi soxazole, Trimethoprim-Sulfamethoxazole(Co-trimoxazole) (TMP-SMX), and Sulfonamidochrysoidine; a tetracycline, such as Demeclocycline, Doxycycline, Minocycline, Oxytetracycline, and Tetracycline; a DHFR inhibitor, such as Aditoprim, Brodimoprim, Iclaprim, Tetroxoprim, and Trimethoprim; a nitroimidazole, such as Metronidazole, Tinidazole, Nimorazole, Dimetridazole, 6-Amino PA824, Ornidazole, Megazol, Azanidazole, and Benznidazole; Tigecycline; Thiamphenicol; a Quinupristin/Dalfopristin combination; Plazomicin, Eravacycline, Mupirocin Sarecycline, Omadacycline, Pretomanid, Lefamulin, Cefiderocol, Ceftazidime, Delafloxacin, Vaborbactam, and Ozenoxacin.

In some embodiments, the antibiotic compound is tobramycin.

In certain embodiments, the compounds described herein above are used in accordance with the present invention to increase sensitivity of bacteria residing in a biofilm to antibiotic treatment. A statistically significant decrease in the minimally effective concentration of an antibiotic agent required to reduce or eliminate a bacterial infection after treatment of a biofilm with the compound of the present invention as compared with a biofilm-based infection prior to treatment is considered as an increase in sensitivity of bacteria residing in a biofilm to antibiotic treatment.

It has been found in accordance with the present invention that the compounds of formula (I), formula (II) and formula (III) suppress prominent virulence determinants (Example 2).

Thus, in an additional aspect, the present invention provides at least one compound selected from a compound of formula (I), a compound of formula (II) and a compound of formula (III), for use in reducing bacterial virulence. Reduction in virulence may be established by measuring a statistically significant reduction in expression or secretion of virulence factors, such as (for the case of pseudomonas) pyocyanin, pyoverdine, elastase (activity of LasB) lipase, rhamnolipids, total protease or chitinase.

Accordingly, in a further aspect, the present invention provides a method of reducing bacterial virulence, the method comprising administering to a subject in need thereof at least one compound selected from a compound of formula (I), a compound of formula (II), and a compound of formula (III).

In some embodiments, the at least one compound is a compound of formula (I) and/or a compound of formula (III).

In some embodiments, the at least one compound is a compound of formula (I) and a compound of formula (III).

In yet an additional aspect, the present invention is directed to a composition comprising at least one compound selected from a compound of formula (I), a compound of formula (II), and a compound of formula (III).

In some embodiments, the at least one compound is a compound of formula (I) and/or a compound of formula (III).

In some embodiments, the at least one compound is a compound of formula (I) and a compound of formula (III).

In certain embodiments, the composition further comprises a pharmaceutically acceptable carrier, i.e. the composition is a pharmaceutical composition.

In certain embodiments, the pharmaceutical composition is in the form of a gel, a stick pill, a rinsing liquid, toothpaste, a tablet, a topical medicament, an oral dentifrice, an injectable composition, an oral tablet, a lozenge, a soft gelatin capsule or an aerosol spray.

Methods for coating a surface with a biologically or pharmaceutically active compound are well known in the art. For example, the non-biological surfaces mentioned above, i.e. the surface of a medical device or a surface of a submerged device, may be coated by blending the compounds described above into film-forming components, and are therefore made into an anti-biofilm coating which can be used to inhibit biofilm formation on the surface of the medical device or submerged object. The film-forming components may comprise one or more resin, such as but not limited to, one or more hydrolysable, soluble or insoluble resins. For example, the resins can be one or more of glyptal resin, acrylic resin, chlorinated rubber resin, epoxy resin, silicone resin, polyester resin, polyurethane resin, fluoropolymer resin, and other resins known to those skilled in the art. The film-forming components can be components of paint, such as a marine paint. The anti-biofilm coating may be in the form of paint.

The term “treating” or “therapy” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

The term “prophylaxis” as used herein refers to means of preventing or delaying the onset of disease and/or symptoms attributed to the disease.

The term “metaphylaxis” or “metaphylactic” as used herein refers to mass medication of a group of animals, in advance of an expected outbreak of disease.

As used herein, the terms “subject” or “individual” or “animal” or “patient” or “mammal,” refers to any subject, particularly a mammalian subject, poultry or fish, for whom diagnosis, prognosis, metaphylactic treatment, prophylactic treatment or therapy is desired, for example, a human or a domesticated mammal such as a pet, farm animal, meat animal, dog, cat, cow, pig, sheep, goat or horse; or poultry and fish.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. In certain embodiments, the pharmaceutical composition is adapted for oral administration.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

According to the present invention, any pharmaceutically acceptable salt of the active agent can be used. Examples of pharmaceutically acceptable salts include, without being limited to, the mesylate salt, the esylate salt, the tosylate salt, the sulfate salt, the sulfonate salt, the phosphate salt, the carboxylate salt, the maleate salt, the fumarate salt, the tartrate salt, the benzoate salt, the acetate salt, the hydrochloride salt, and the hydrobromide salt.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

The invention will now be illustrated by the following non-limiting examples.

### EXAMPLES

### Materials and Methods

100 bacterial isolates obtained from several coral species were screened for their anti-biofilm activity and abilities to inhibit quorum sensing using different bioreporter strains. Active compound identification was performed by separation, reverse thin layer chromatography, followed by preparative HPLC, and finally using MS and NMR spectroscopy. These techniques were used to elucidate the main active structures identified as 1,1′-Biindolyl (NN) and 2-(indolin-2-yl)-1H-indole (DIV) (Table 1).

¹H NMR and ¹³C NMR spectra and two-dimensional NMR analysis were used to validate the structure and purity of DIV. The chemical shifts were found to be in a good agreement with the results reported by Somei et al. (1997).

¹H-NMR (DMSO 400 MHz) δ 3.1 (1 H, dd, J=154 and J=9.2 Hz), 3.7 (1 H, dd, J=15.4 and 9.2 Hz), 5.8 (1 H, dt, J=2.7 and 9.2 Hz), 6.0 (1 H, d, J=2.7 Hz), 6.3 (1 H, d, J=2.2 Hz), 6.5 (1 H, ddd, J=7.3, 6.5, and 1.0 Hz), 6.5 (1H, d, J=7.3 Hz), 6.9 (1H, ddd, J=7.3, 6.5, and 1.0 Hz), 6.9 (1H, t, J=7.3 Hz), 7.0 (1H, ddd, J=7.3, 6.5, and 1.0 Hz), 7.04 (1H, d, J=7.3 Hz), 7.3 (1H, dd, J=7.3 and 1.0 Hz), 7.4 (1H, d, J=7.3 Hz), 11.05 (1H, br s).

¹³C NMR (DMSO 400 MHz): δ 140.0, 130.0, 128.0, 127.8, 125.6, 125.3, 124.5, 122.6, 122.0, 120.0, 118.9, 118.3, 116.7, 111.5, 59.8, 38.0.

EI-MS m/z: 235 (M+H)⁺.

We have also synthesised DIV to confirm its chemical purity and structural identity. The ¹H NMR and ¹³C NMR spectra of the synthesised DIV were found in a good agreement with the above spectra of the natural DIV compound.

NN was purchased from MolPort, Lacplesa iela 41, Riga, LV-1011, Latvia.

In addition, we tested the commercially available anti-cancer compound di(1H-indol-3-yl) methane (hereinafter, “DIM”) (Table 1), which was purchased from Sigma Aldrich.

### Example 1. Biofilm Inhibiting Properties of NN and DIV

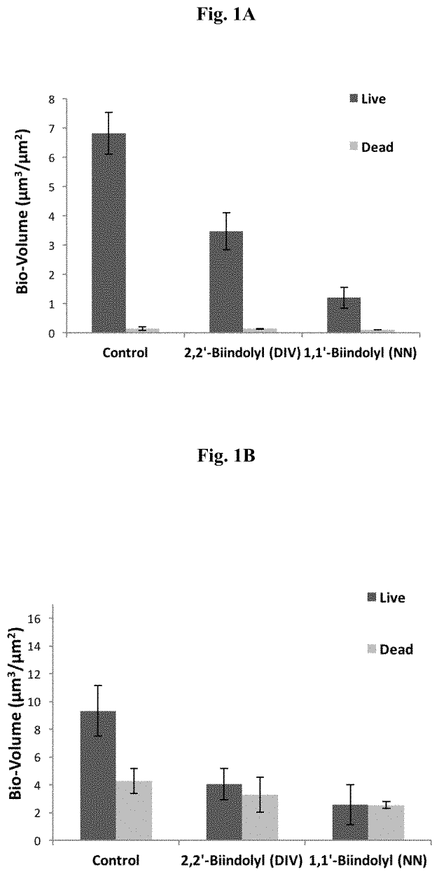

Bioflims of P. aeruginosa and A. baumanii that were developed on glass slides were determined using confocal scanning laser microscopy (CSLM) (FIG. 1). Accordingly, the density of the biofilms prior to and following treatment by NN and DIV was measured. Both treatments resulted in a reduction of density of attached cells, though NN was the most effective for P. aeruginosa. DIV and NN treatment showed smaller effects on density of A. baumanii model strain. Both treatments resulted in negligible mortality of the bacterial cells as they did not differ from the control.

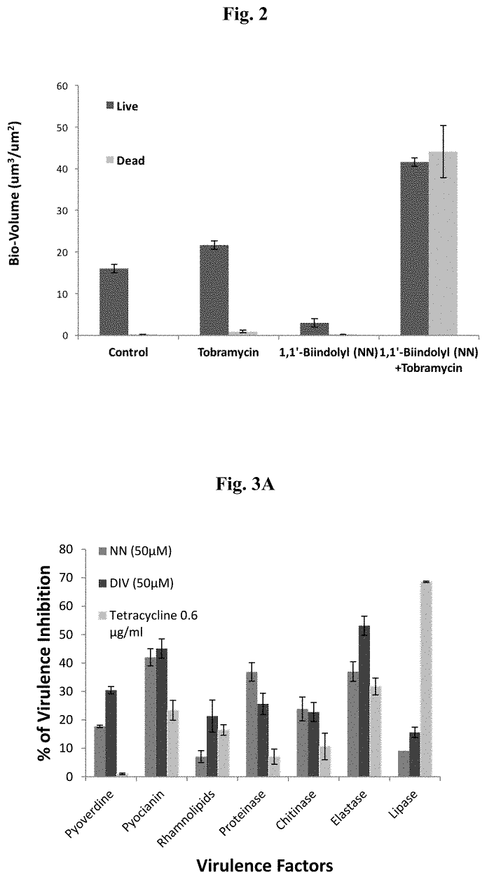

Dynamic growth conditions in flow cell systems are considered to be representative of the real conditions in humeral tissues, where the pathogen thrives in enriched settings. An investigation of the effects of the inhibitor compounds on the bacteria in terms of destruction of already-structured biofilm showed that both compounds had similarly broad anti-biofilm effects on P. aeruginosa PA01 mature biofilm (data not shown). Once the efficiency of the new compounds was tested, we proceeded to assess the efficacy of adding antibiotic treatment to the novel compounds to eradicate biofilm (FIG. 2). The P. aeruginosa biofilm that had been treated by the antibiotic alone showed little loss of biofilm. When treated by NN alone the biofilm was reduced but not eradicated. On the other hand, when treated by both the antibiotic and the NN, the biofilm was eradicated and the bacterial cells died.

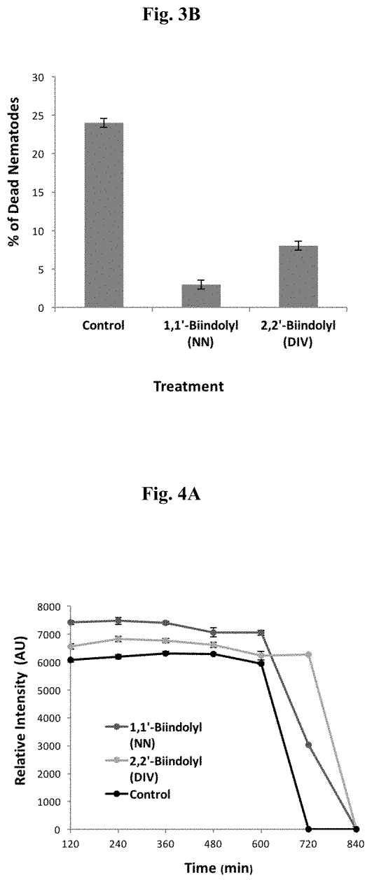

Driven by an arsenal of virulence factors added to the biofilm mode of growth, P. aeruginosa PA01 pathogenesis depends on the type of the infection. Therefore, both compounds were tested for their abilities to suppress prominent virulence determinants (FIG. 3A). Quantization of extracellular virulence factors (pyocyanin pyoverdine, elastase (activity of LasB) lipase, rhamnolipids, total protease and chitinase production by P. aeruginosa PAO1 cell-free culture, was carried out using spectrophotometry. The virulence factors tested, were affected differently by the two compounds. Most of the virulence factors were reduced significantly following exposure of the cells to the two compounds. In addition, we tested NN and DIV on the nematode Caenorhabditis elegans infected by Pseudomonas aeruginosa. The nematode C. elegans is often used as a model for host pathogen interaction in higher multicellular organisms. Measuring nematode survivorship following exposure to P. aeruginosa in the absence and presence of modulating compounds, therefore, provides a good model for assessing the effectiveness of the novel compounds on the pathogenesis of the bacteria. In our experiments the improved survivorship of C. elegans following exposure to the pathogen in conjunction with our compounds showed that pathogenesis of P. aeruginosa is reduced by both of the compounds tested though NN proved to be more effective as it significantly increased survivorship of the nematodes (FIG. 3B).

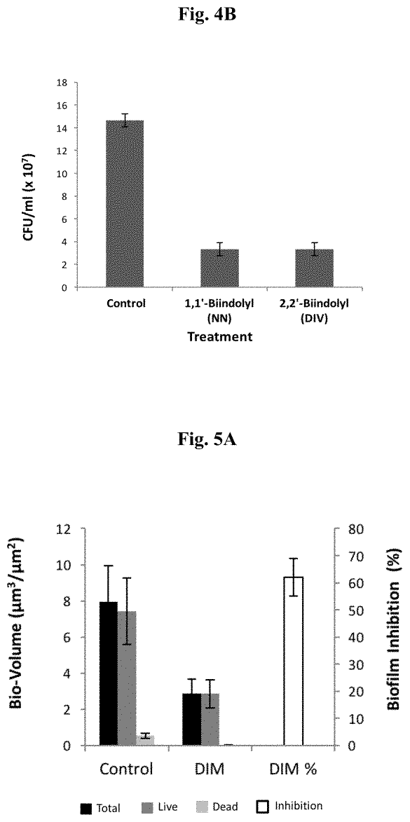

In order to test the possible effects of these compounds on higher organisms, we tested NN and DIV on A549 Human Lung Cell line infected by P. aeruginosa. Additional tests were carried out for NN and DIV to assess the effect of this compound to enhance the survival of A549 cells during infection with P. aeruginosa PA01. As indicated by greater calcein expression, infection with the pre-treated bacteria suspended the cytotoxicity effect and apoptosis killing in A549 during the incubation (FIG. 4A). Due the fact that adherence to a humeral cell is considered a crucial step in the bacterial infection initiation process, P. aeruginosa PA01 pre-treated with DIV or NN was tested for its adherence potency to lung epithelial cells A549. When compared to release following culture of the bacteria in DMSO, culture in either of the compounds (NN or DIV) resulted in similar percentage of release of the pathogenic P. aeruginosa PA01 from infected cells cultured in the 96-well plate (FIG. 4B).

### Example 2. Biofilm Inhibiting Properties of 3,3′-Diindolylmethane (DIM)

In addition, we tested the anti-biofilm properties of the well-studied anti-cancer compound 3,3′-Diindolylmethane (DIM), a metabolite found in cruciferous vegetables. Inhibition of biofilm establishment by DIM was investigated with different clinical pathogens under static conditions. Biofilm formation by A. baumannii and P. aeruginosa was further tested in a dynamic flow-cell system, in which biofilm inhibition levels of 86% and 76% were obtained, respectively. Combined treatment comprising tobramycin and DIM showed significant biofilm formation inhibition percentages of 94% that manifested in the almost complete eradication of bacteria. Moreover, the results also suggest that DIM can potentially inhibit the secretion of a distinctive virulence factor by P. aeruginosa. Further examination of the hypothesized synergistic effect obtained by combining conventional antibiotics with the DIM compound may offer a promising strategy for the eradication of biofilm complexes.

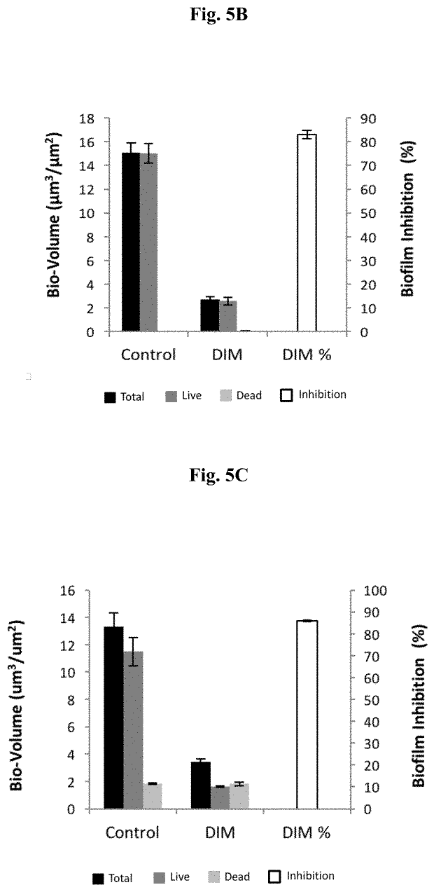

The current study investigated the influence of DIM on the biofilm formation process and on the destruction of existing biofilms of several pathogenic gram-negative bacterial strains. The introduction of DIM to bacterial cultures led to the formation of substantially reduced biofilms by A. baumannii, S. marcescens, P. stuartii, and P. aeruginosa PA01 when compared to the thick, live biofilms of the control samples (FIGS. 5A-5D).

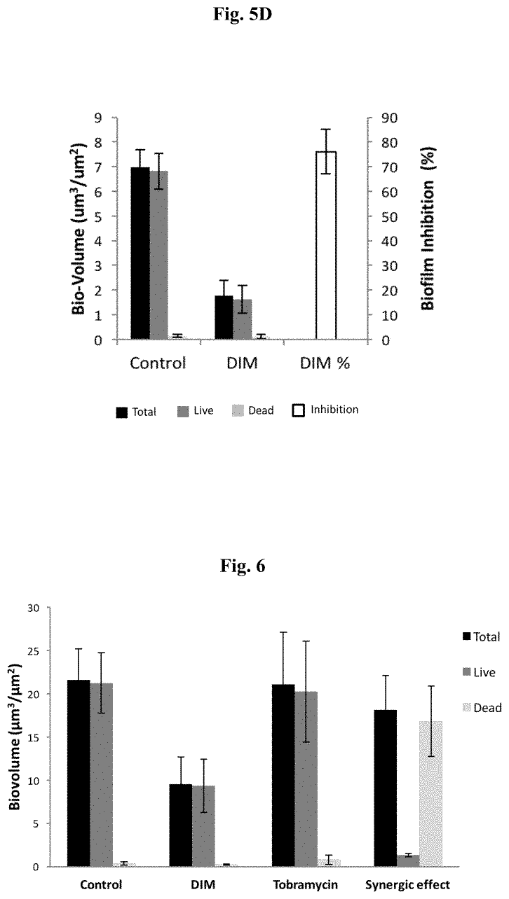

To investigate the combined destructive effect of the biofilm inhibitor compound and an antibiotic, P. aeruginosa PA01 was cultured for 72 h in the continuous flow system until a mature biofilm had been established. Immediately after its establishment, the biofilm was challenged with DIM and/or the antibiotic tobramycin, the known activity of which is protein synthesis inhibition (FIG. 6). Biofilms formation after 48 h showed that addition of only tobramycin resulted in dense biofilm similar to that of the control. In contrast, the addition of DIM alone to the medium supplied to the biofilm cells led to the destruction of existing, stable biofilm—and thus, to a more sparsely distributed architecture—possibly by enhancing detachment of the biofilm from the surface, which resulted in the exposed planktonic bacteria being washed away. In stark contrast, the synergic DIM-tobramycin treatment almost completely eradicated the biofilm, a difference clearly manifested in the number of dead cells.

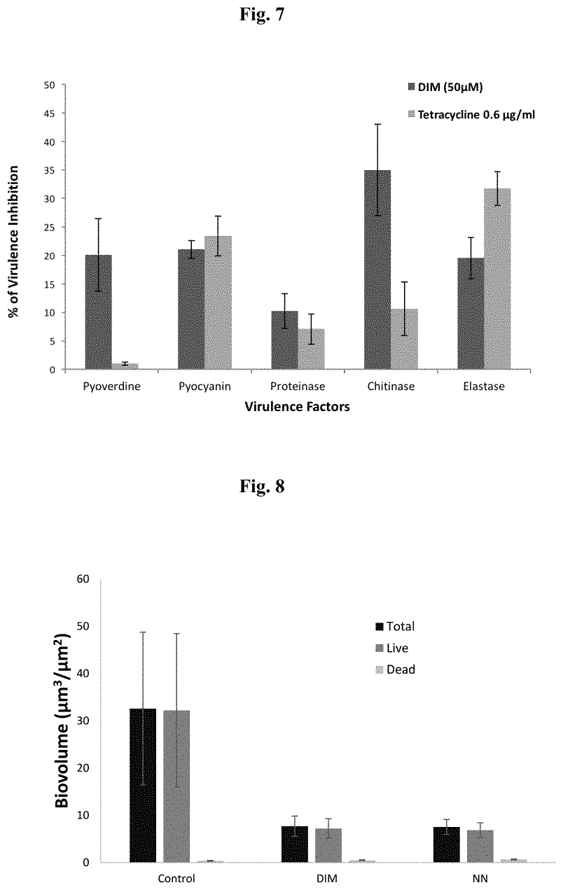

DIM exhibits a potential to interfere with the cellular pathways involved in virulence factor production. The basis of P. aeruginosa pathogenicity is an arsenal of virulence determinants designed for survival and proliferation in the host that enable bacterial invasion and the subsequent establishment of infection. The success of this microorganism is largely due to its ability to form intractable biofilms and to produce myriad virulence factors controlled by a quorum-sensing system. A significant reduction of 35% in chitinase production was observed in the presence of 50 μM DIM, while smaller reductions of 20%, 21%, 10% and 19% were observed in pyoverdine, pyocyanin, protease and elastase, respectively (FIG. 7).

### Example 3. Biofilm Inhibiting Properties of DIM and NN on Plant-Associated Bacteria

Erwinia carotovora was cultured in glass-bottomed 96 well plates for 18 h in static conditions. The growth medium was provided with 50 μM DIM or NN and the resulted biofilms was investigated using CSLM.

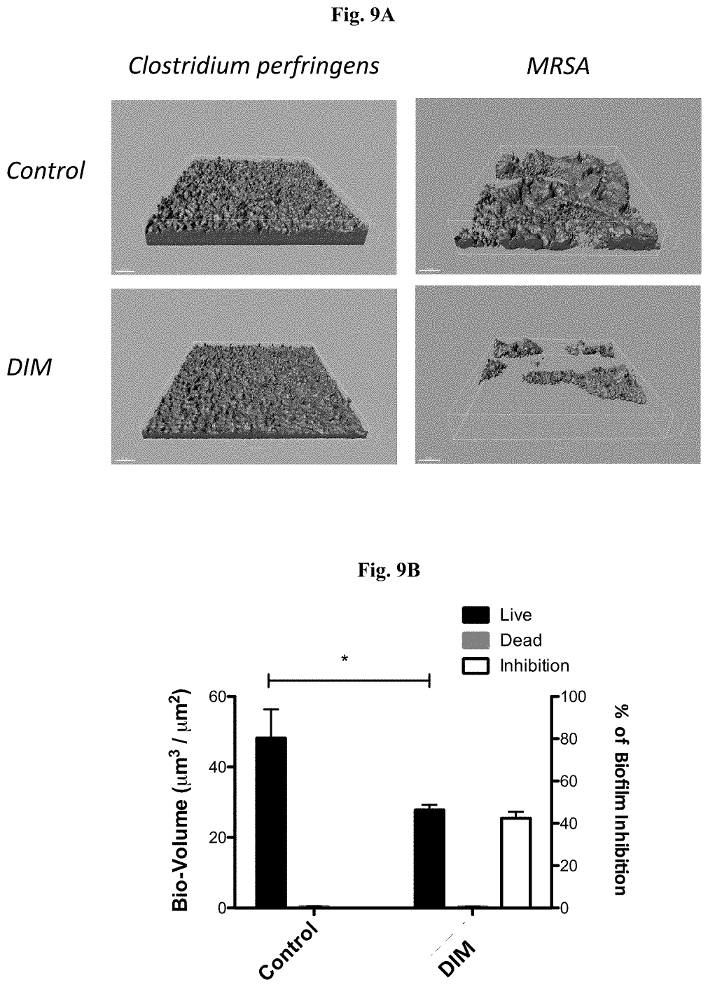

Both treatments resulted in a significant reduction of density of attached cells (FIG. 8).

### Example 4. Assessing Biofilm Formation of Model Gram Positive Bacteria

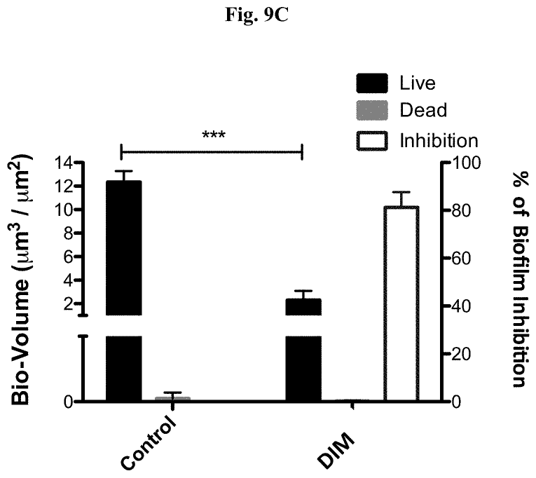

The anti-biofilm properties of DIM were additionally tested on representative Gram-positive pathogenic bacteria strains including Clostridium perfringens and Staphylococcus aureus. Biofilms of Clostridium perfringens and Staphylococcus aureus were cultivated in a continuous flow-cell system, in flow chambers supplied with 1% TSB (Difco Luria-Bertani medium, BD, France). The flow cell was inoculated with an overnight culture diluted with 0.9% NaCl to an OD₆₀₀ of 0.1. The medium was continually pumped at a constant rate of 3 ml h⁻¹ at 37° C. for the duration of the experiment using a peristaltic pump. The activity of DIM was evaluated by constantly treating one of the biofilms with 50 μM of the compounds supplemented to the medium, while for the control, another biofilm of the same bacteria was treated with an equivalent amount of DMSO. Treatment was given for 48 h, after which time, biofilms were stained with LIVE/DEAD™ BacLight™ bacterial viability staining kit (Molecular Probes Inc., Eugene, Oreg., USA) and their thickness was measured and assessed using a CSLM and the IMARIS software s3 (Bitplane AG, Zürich, Switzerland). The densities and viabilities of the bacterial biofilms were assayed by staining with SYTO 9 (Molecular Probes Inc., Eugene, Oreg., USA), which stains live cells green, and propidium iodide (Molecular Probes Inc., Eugene, Oreg., USA), which stains dead cells red. This method enabled visualizing the live and dead cells using CSLM (FIG. 9). A comparison of biofilm density following treatment with 50 μM of DIM showed that DIM led to a reduction in the density of attached cells, the degree to which this occurred varied depending on bacterial strain. C. perfringens biofilm was inhibited by 45% while S. aureus biofilm was inhibited by 82% (FIGS. 9B and 9C, respectively). As can be seen in FIG. 9, DIM treatment caused only negligible bacterial cell mortality in all bacterial strains.

The reduction in the structure or densities of attached cells as a result of the application of DIM will therefore enable other antimicrobial (antibiotic) agents to penetrate developed biofilms.

### REFERENCES

Brenda V Bertinetti, M Alejandra Rodriguez, Alicia M Godeas and Gabriela M Cabrera. 1H,1′H-[3,3′]biindolyl from the terrestrial fungus Gliocladium catenulatum. The Journal of Antibiotics (2010) 63, 681-683.

Somei M; Hayashi, H.; Ohmoto, S. “Reduction of indigo: Simple syntheses of 3-acetoxy-, 1-acetyl-2.3-dihydro-, 3-acetoxy-3′-acetyl-, 3-acetoxy-1,3′-diacetyl-2,2′-bisindoles, and 2,2′-bisindole”, Heterocycles 1997, 44 (1), pp. 169-176.

Wu H, Claus Moser, Heng-Zhuang Wang, Niels Høiby and Zhi-Jun Song. “Strategies for combating bacterial biofilm infections”. International Journal of Oral Science (2014) 7, 1-7.

Zhang L, Jing Xia, Qinghai Li, Xihong Li, and Shaowu Wang “Fast Synthesis of Hydrazine and Azo Derivatives by Oxidation of Rare-Earth-Metal-Nitrogen Bonds”, Organometallics 2011, 30, 375-378.

1. A pharmaceutical composition comprising 2-(indolin-2-yl)-1H-indole and/or 1,1′-biindole, and a pharmaceutically acceptable carrier.

2. The pharmaceutical composition of claim 1, wherein said composition is a gel, a stick pill, a rinsing liquid, a toothpaste, a tablet, a topical medicament, an oral dentifrice, an injectable composition, an oral tablet, a lozenge, a soft gelatin capsule or an aerosol spray.

3. The pharmaceutical composition of claim 1, further comprising at least one antibiotic or antibacterial agent.

4. A method for inhibiting formation of biofilm by bacteria on a surface or disrupting existing biofilm formed by bacteria on a surface, comprising contacting said surface or said existing biofilm with an effective amount of the pharmaceutical composition of claim 1, wherein said surface is a surface of a mammalian cell, tissue, organ, system, or structure.

5. The method of claim 4, wherein said bacteria are gram-negative bacteria.

6. The method of claim 5, wherein said Gram-negative bacteria are selected from the group consisting of acetic acid bacteria; Acinetobacter; Bdellovibrio; Escherichia coli (E. coli), Salmonella, Shigella, and other Enterobacteriaceae; Erwinia; Haemophilus influenzae; Helicobacter; Klebsiella pneumoniae; Legionella; Moraxella; Neisseria gonorrhoeae; Neisseria meningitidis; Proteus mirabilis; Providencia; Pseudomonas; Serratia; Stenotrophomonas, and more specifically from Pseudomonas aeruginosa, Acinetobacter baumannii, Serratia marcescens, Providencia stuartii, and Erwinia carotovora.

7. The method of claim 4, wherein said bacteria are gram-positive bacteria.

8. The method of claim 7, wherein said Gram-positive bacteria are selected from the group consisting of Streptococcus, Staphylococcus, Corynebacterium, Listeria, Bacillus, Clostridium, Catabacter hongkongensis, Mycobacterium, Mycoplasma, Enterococci, and Actinomyces.

9. The method of claim 4, wherein said surface is a surface of a cell, tissue, or structure of the gastrointestinal tract, the respiratory system, the skin, or of an epithelium.

10. The method of claim 4, wherein said surface is a surface of a medical device intended for insertion into a subject's body, such as a pacemaker, pacemaker leads, catheter or stent.

11. The method of claim 4, for prophylaxis, metaphylaxis, or therapy or an infectious disease caused by said bacteria in said biofilm.

12. The method of claim 4, further comprising contacting said surface with at least one antibiotic or antibacterial agent.

15. A medical device coated, washed, or rinsed with the pharmaceutical composition of claim 1.

16. The medical device of claim 15, which is a pacemaker, pacemaker leads, catheter or stent.
